(19) United States
(12) Patent Application Publication
(10) Pub. No.: US 2013/0289636 A1
(43) Pub. Date: Oct. 31, 2013

(54) TIMED IMPLANTABLE THERAPY DELIVERY DEVICE

(71) Applicants: Mustafa Karamanoglu, Fridley, MN (US); Vincent E. Splett, Apple Valley, MN (US); Teresa A. Whitman, Dayton, MN (US)

(72) Inventors: Mustafa Karamanoglu, Fridley, MN (US); Vincent E. Splett, Apple Valley, MN (US); Teresa A. Whitman, Dayton, MN (US)

(21) Appl. No.: 13/652,815

(22) Filed: Oct. 16, 2012

Related U.S. Application Data
(60) Provisional application No. 61/640,464, filed on Apr. 30, 2012.

(51) Int. Cl. A61N 1/39; A61N 1/362

(52) U.S. Cl. 607/4

(57) ABSTRACT

Pulseless electrical activity (PEA) is reduced or eliminated. A medical electrical lead is implanted to deliver high voltage therapy to a fibrillating heart. Another medical electrical lead delivers electrical stimulation through an electrode proximate phrenic nerve tissue in response to the delivery of high voltage therapy to the fibrillating heart.

### TECHNICAL FIELD

The disclosure relates generally to implantable medical devices and, more particularly, to a method and apparatus for a timed therapy delivery device for treatment of pulseless electrical activity.

### BACKGROUND

Some types of implantable medical devices, such as cardiac pacemakers or implantable cardioverter defibrillators (ICDs), provide therapeutic electrical stimulation to a heart of a patient via electrodes on one or more implantable medical electrical leads. The therapeutic electrical stimulation may be delivered to the heart in the form of pulses or shocks for pacing, cardioversion or defibrillation.

Some patients with ICDs have a potential risk of experiencing pulseless electrical activity (PEA) due to due to long duration of fibrillation caused by failure to defibrillate, or failure of defibrillation therapy to restore electro-mechanical coupling and recovery of sufficient contraction force. PEA involves persistent electrical activity in the heart without associated mechanical contraction. Lack of mechanical contraction to generate a palpable pulse reduces cardiac output that can result in sudden death. It is therefore desirable to develop a therapy delivery system that is able to deliver therapy to a fibrillating heart and addresses any resultant PEA.

### BRIEF DESCRIPTION OF THE DRAWINGS

FIG. 1A is a schematic view of an implantable medical device (IMD) system for delivering phrenic nerve stimulation and/or cardiac therapy according to one or more embodiments.

FIG. 1B is a schematic view of a back side of a patient that includes a vertebral column, spinal nerves with electrodes positioned near the thoracic vertebrae levels.

FIG. 1C is a schematic side view of the spinal cord shown in FIG. 1B along the mid-axillary line in which each segment function is displayed.

FIG. 2 is a schematic view of an IMD system for delivering phrenic nerve stimulation according to an alternative embodiment.

FIG. 3 is a schematic view of an IMD system for delivering phrenic nerve stimulation according to another alternative embodiment.

FIG. 4 is a functional block diagram of an IMD that may be associated with any of the leads and implant locations shown in FIGS. 1A through 3.

FIG. 5 is a flow diagram of a method for delivering a timed therapy before delivery of therapeutic shock is delivered.

FIG. 6 is a flow diagram of a method for delivering therapeutic shock followed by a timed therapy to address pulseless electrical activity.

FIG. 7 is a flow diagram of a method that involves delivering phrenic nerve stimulation, a therapeutic shock followed by electrical stimulation to elicit a cough to address pulseless electrical activity.

### DETAILED DESCRIPTION

The following description of the preferred embodiments is merely exemplary in nature and is in no way intended to limit the invention, its application, or uses. For purposes of clarity, similar reference numbers are used in the drawings to identify similar elements.

The present disclosure reduces incidence of pulseless electrical activity (PEA) in patients with diminished cardiac function. Reduction in PEA is accomplished by delivering electrical stimulation to phrenic nerve tissue in order to cause a patient to inspire and expire. Immediately after taking a deep breath, electrical stimulation is delivered to abdominal/intercostal tissue, which prompts the patient to cough. Sequentially combining phrenic nerve stimulation with abdominal/intercostal stimulation moves oxygenated blood out of the thorax, reduces the size of the ventricles, and perfuses the coronaries. Reduction in cardiac size and increased perfusion results in improved defibrillation efficacy and assists in returning to spontaneous circulation post-shock.

Although the following description generally relates to the use of a first implantable medical device (e.g. implantable cardioverter defibrillators (ICD) in combination with a second IMD (e.g. neurostimulator), it is readily understood that the broader aspects of the present disclosure are applicable to other types of IMDs. In particular, the present disclosure may be applied to a single implantable medical device (IMD) that includes a neurological module (e.g. computer instructions) and a cardiac module executed by a microprocessor, all of which is housed in a single housing. It is generally understood that the disclosure may also be implemented with more or less medical electrical leads than that which is depicted in the figures.

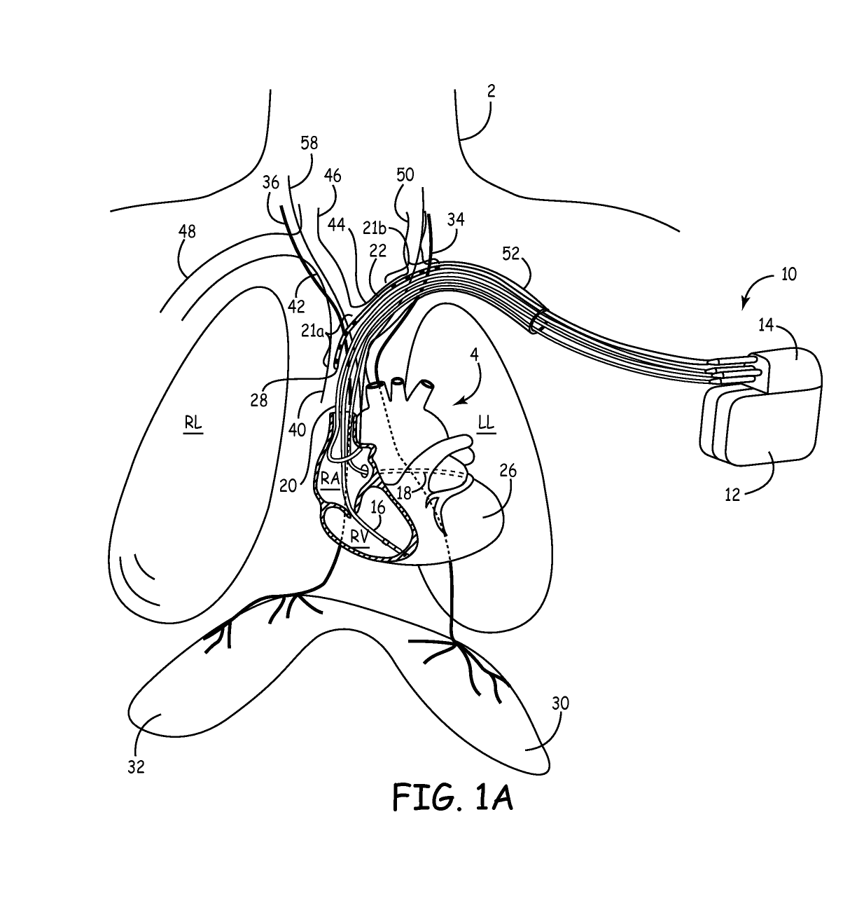

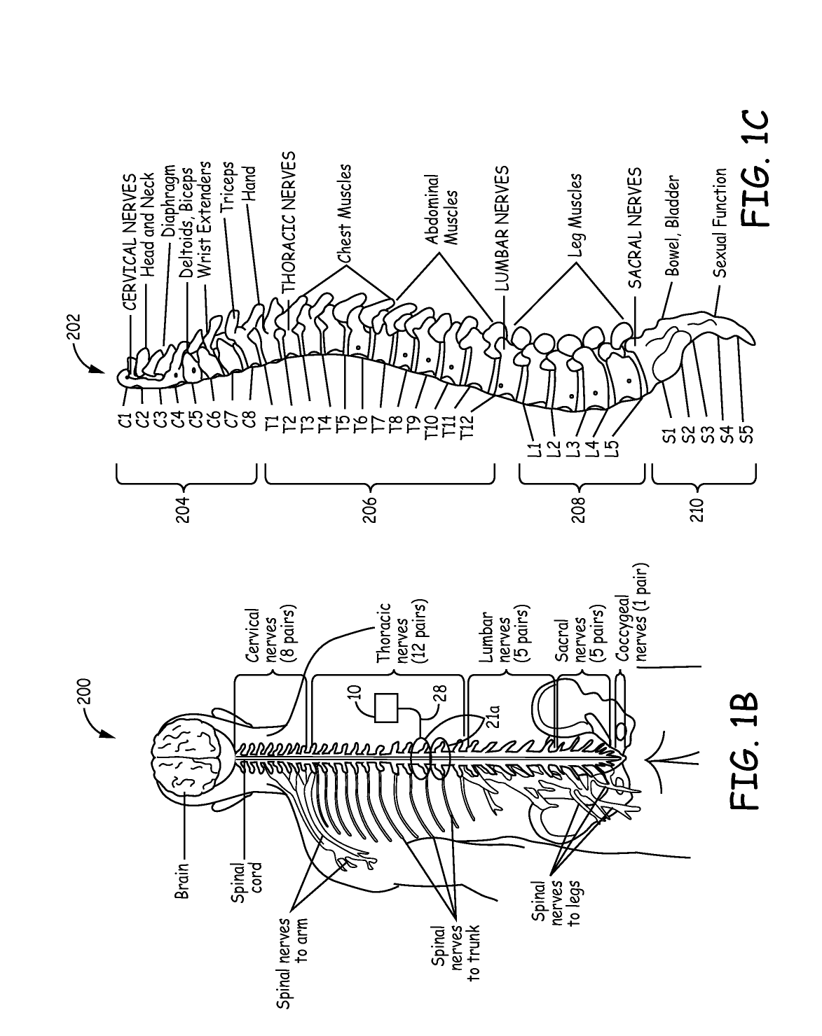

FIG. 1A is a schematic view of an IMD system 10 that reduces incidence of pulseless electrical activity (PEA) in patients with reduced cardiac function. An exemplary cardio-neurostimulator may be seen with respect to US Publication No. 2010/0114196 A1 filed on Jan. 30, 2009, which is incorporated by reference in its entirety. IMD 10 includes a housing 12 enclosing electronic circuitry (not shown) included in IMD 10 and a connector block 14 having a connector bore for receiving two or more medical electrical leads 16, 18, 20, 22. Medical electrical leads 16, 18, 20, 22 include one or more electrodes that are electrically connected with IMD internal electronic circuitry. Medical electrical leads 16, 18, 20 are cardiac leads. Lead 16 is configured and placed to deliver defibrillation shock(s) while leads 18, 20 are configured to deliver pacing pulses to the cardiac tissue.

In FIG. 1A, leads 16, 18, 20 extend into the heart 4 of the patient 2 to sense electrical activity (electrical cardiac signals) of heart 4 and/or deliver electrical stimulation (cardiac therapy) to heart 4. In particular, right ventricular (RV) lead 16 extends through one or more veins (not shown), superior vena cava (not shown), and right atrium 30, and into right ventricle 24. Left ventricular (LV) coronary sinus lead 18 extends through one or more veins, the vena cava, right atrium (RA), and into coronary sinus to a region adjacent to the free wall of left ventricle 26 of heart 4. Right atrial (RA) lead 16 extends through one or more veins and the vena cava, and into right atrium 30 of heart 4. In other examples, IMD 10 and, more particularly, the cardiac therapy module of IMD 10, may deliver stimulation therapy to heart 4 by delivering stimulation, via the cardiac therapy module, to an extravascular tissue site in addition to or instead of delivering stimulation via electrodes of intravascular leads 16, 18, 20 22. In such examples, therapy system or IMD 10 includes one or more extravascular leads mechanically and electrically connected to IMD 10.

The cardiac therapy module may sense electrical signals attendant to the depolarization and repolarization of heart 4 via electrodes (not shown in FIG. 1A) coupled to at least one of the leads 16, 18. These electrical signals within heart 4 may also be referred to as cardiac signals or electrical cardiac signals. In some examples, the cardiac therapy module provides pacing pulses to heart 4 based on the electrical cardiac signals sensed within heart 4. The configurations of electrodes used by the cardiac therapy module for sensing and pacing may be unipolar or bipolar. The cardiac therapy module may also provide defibrillation therapy and/or cardioversion therapy via electrodes located on at least one of the cardiac leads such as lead 16 and one or more electrodes on housing 12 of IMD 10. IMD 10 may detect arrhythmia of heart 4, such as fibrillation of ventricles 24 and 26, and deliver defibrillation therapy to heart 4 in the form of electrical pulses via one or more of leads 16, 18, and 20. In some examples, the cardiac therapy module may be programmed to deliver a progression of therapies, e.g., pulses with increasing energy levels, until a fibrillation of heart 4 is stopped. IMD 10 detects fibrillation employing one or more fibrillation detection techniques known in the art.

Lead 20 is placed near the left and/or right phrenic nerve 34, 36 using any known means. In FIG. 1A, the left phrenic nerve 34 and the right phrenic nerve 36 are shown innervating the respective left diaphragm 30 and right diaphragm 32. The anatomical locations of the left phrenic nerve 34, the right phrenic nerve 36 and other anatomical structures shown schematically in the drawings presented herein are intended to be illustrative of the approximate and relative locations of such structures. These structures are not necessarily shown in exact anatomical scale or location. Left phrenic nerve 34 is shown schematically to extend in close proximity to the left internal jugular vein (LJV) 50, the left subclavian vein (LSV) 52, and the left innominate vein (LIV) 44, also referred to as the left brachiocephalic vein.

The anatomical location of the right phrenic nerve 36 is shown schematically to extend in close proximity to the right internal jugular vein (RJV) 46, the right subclavian vein (RSV) 48, the right innominate vein (RIV) 42 (also referred to as the right brachiocephalic vein), and the superior vena cava (SVC) 40.

Lead 22 is a multipolar neurological lead carrying proximal electrodes 21a spaced proximally from distal electrodes 21b, positioned at or near the distal end 18 of lead 16. Skilled artisans appreciate that other embodiments can rely on any one of the medical electrical leads described and/or incorporated herein, or other suitable leads. In one or more embodiments, at least one proximal bipolar pair of electrodes is provided for stimulating the left phrenic nerve 34 and at least one distal bipolar pair of electrodes is provide for stimulating the right phrenic nerve 36. In various embodiments, two or more electrodes may be spaced apart along the lead body, near the distal tip 28 of lead 22, from which at least one pair of electrodes is selected for delivering stimulation to the right phrenic nerve 36. Additionally, two or more electrodes may be positioned along spaced apart locations proximally from the distal electrodes 21a from which at least one pair of electrodes 21b is selected for delivering stimulation to the left phrenic nerve 34.

Lead 22 includes an elongated lead body, which may have a diameter in the range of approximately 2 French to 8 French, and typically about 4 French to about 6 French. The lead body carries the electrodes 21a and 21b which are electrically coupled to electrically insulated conductors extending from respective individual electrodes 21a and 21b to a proximal connector assembly adapted for connection to IMD connector block 14. Lead 22 may be provided with a fixation element for placing or connecting the lead 22 to a desired implant location. Exemplary leads that can be useful for the present disclosure include U.S. Pat. No. 5,922,014, U.S. Pat. No. 5,628,778, U.S. Pat. Nos. 4,497,326, 5,443,492, U.S. Pat. No. 7,860,580 or US Patent Application 20090036947 filed Apr. 30, 2008 such that electrodes are added and/or spaced apart in a manner similar to that disclosed in the figures of the present application, all of listed patents and applications are incorporated by reference in their entirety. Additional lead and electrode configurations that may be adapted for use with the present disclosure by adjusting lead shape, length, electrode number and/or electrode to effectively provide phrenic nerve stimulation as described herein are generally disclosed in U.S. Pat. No. 7,031,777, U.S. Pat. No. 6,968,237, and US Publication No. 2009/0270729, all of which are incorporated herein by reference in their entirety.

In one embodiment, distal tip 28 of lead 22 is advanced to a location along the RIV 42 and further along the RSV 48 or the RJV 46 to position distal electrodes 20 in operative relation to right phrenic nerve 36 for delivering stimulation pulses to nerve 36 to activate the right diaphragm 32. The proximal electrodes 21a may be appropriately spaced from distal electrodes 21b such that proximal electrodes 21a are positioned along the LIV 44 and/or along the junction of the LSV 52 and LJV 50 for delivering stimulation pulses to the left phrenic nerve 34 to activate the left diaphragm 30.

In various embodiments, lead 22 may carry four or more electrodes spaced at selected distances to provide at least one pair near a distal lead tip 28 for right phrenic nerve stimulation and at least one pair more proximally for left phrenic nerve stimulation. In other embodiments, lead 22 may carry multiple electrodes spaced equally along a portion of the body of lead 22 such that any pair may be selected for right phrenic nerve stimulation and any pair may be selected for left phrenic nerve stimulation based on the relative locations of the electrodes from the nerves. Furthermore, it is recognized that in some embodiments, stimulation of only one of the right or left phrenic nerve may be required and an appropriate number and location of electrodes may be provided along lead 22 for such purposes. Additionally, lead 22 can be placed endovascularly near the phrenic nerve or extravascularly using any known methods. A cuff electrode can be used to extravascularly connect the electrode to nerve tissue.

To elicit a cough from the patient, lead 22 or another medical electrical lead (e.g. neurological lead) that includes one or more electrodes is placed near or to nerve tissue proximate a spinal column for a human patient 200, as depicted in FIGS. 1B-1C, which is briefly described below. The spinal column includes a vertebra column 202. The vertebral column extends along eight cervical (C1-C8) vertebrae 204, twelve thoracic vertebrae (T1-T12) 206, five lumbar vertebrae (L1-L5) 208 and five sacral vertebrae (S1-S5) 210. The vertebral column 202 is innervated with cervical nerves, thoracic nerves, lumbar nerves, sacral nerves, and coccygeal nerves, each of which generally extends to the brain. Cervical nerves (eight pairs) extend along C1-C8 and allow signals to be transmitted between the brain and the neck, diaphragm, deltoids, biceps, wrist, triceps and hands. Thoracic nerves extend between the costae or ribs to the brain. Thoracic nerves (twelve pairs) transmit signals between the brain and the chest muscles and the abdominal muscles. Lumbar nerves (five pairs) extend from the brain to the legs. Sacral nerves (five pairs) extends to the brain and controls the bowel, bladder and sexual function.

Numerous ways exist in order to elicit a cough from a patient. One way to elicit a cough involves the use of a single neurological lead. For example, lead 22 could be placed near both phrenic nerve tissue and thoracic spinal nerve tissue. Electrical stimulation of the thoracic spinal nerve tissue induces a forced contraction causing a cough. The electrical stimulation delivered to phrenic nerve tissue can be delivered at a different or about the same amplitude as that which is provided for AIS. Alternatively, a separate neurological lead can be placed alongside thoracic nerve tissue in order to elicit a cough. The intercostal muscles, located between the ribs, include the internal intercostal muscles and the external intercostal muscles. The thoracic cavity can expand through raising the rib cage with external intercostal muscles thereby causing inspiration of air. Internal intercostal muscles are located deep to inside the external intercostal muscles, and contraction of the internal intercostal muscles pull the ribs together to increase intrathoracic pressure and force air out of the lungs.

For example, FIGS. 1B-1C generally disclose placement of medical electrical lead electrodes in a patient 200 in order to generate an expiratory function such as a cough. As shown in FIG. 1B, to generate a cough, electrodes are positioned along a vertebral column 202 and rib cage, between the ribs, at T1 through T12 but more preferably at T9-T10 which resulted in greatest airway pressures. One or more electrodes can be positioned between two adjacent ribs and within about 3 cm externally (distally) of the corresponding neuroforamen from which the spinal nerve emerges. In the illustrated example, the electrodes are placed near the inferior margin of each nerve. The inferior margin of each nerve is in close proximity to the intercostal nerve. The electrode can be positioned within about 5 cm externally of the neuroforamen. Alternatively, each electrode is positioned within about 2 cm externally of the neuroforamen. Skilled artisans appreciate that adjustments can be made as to the number of electrodes employed, and/or distance between placement of the electrode from the nerve and/or muscle tissue.

Numerous other electrode(s) placement can also be implemented in order to deliver AIS or SCS stimulation. For example, subcutaneous defibrillation locations can be used to deliver more localized stimulation to the intercostals and abdominal muscles as shown and described relative to U.S. Pat. No. 7,769,452 B2 to Ghanem et al. issued Aug. 3, 2010, and incorporated by reference in its entirety herein. US Pregrant Publication No. 2008/0051581A1 filed Aug. 28, 2006 provides another exemplary placement of the electrodes on a lead, which is incorporated by reference in its entirety.

In one or more other embodiments, a first IMD (e.g. implantable cardioverter defibrillator (ICD) etc.) is implanted to deliver cardiac therapy to the heart and a second IMD is implanted to deliver electrical stimulation to phrenic nerve tissue. An exemplary neurostimulator may be seen with respect to U.S. patent application Ser. No. 11/810,941 filed on Jun. 7, 2007, and assigned to the assignee of the present invention, the disclosure of which is incorporated by reference in its entirety herein.

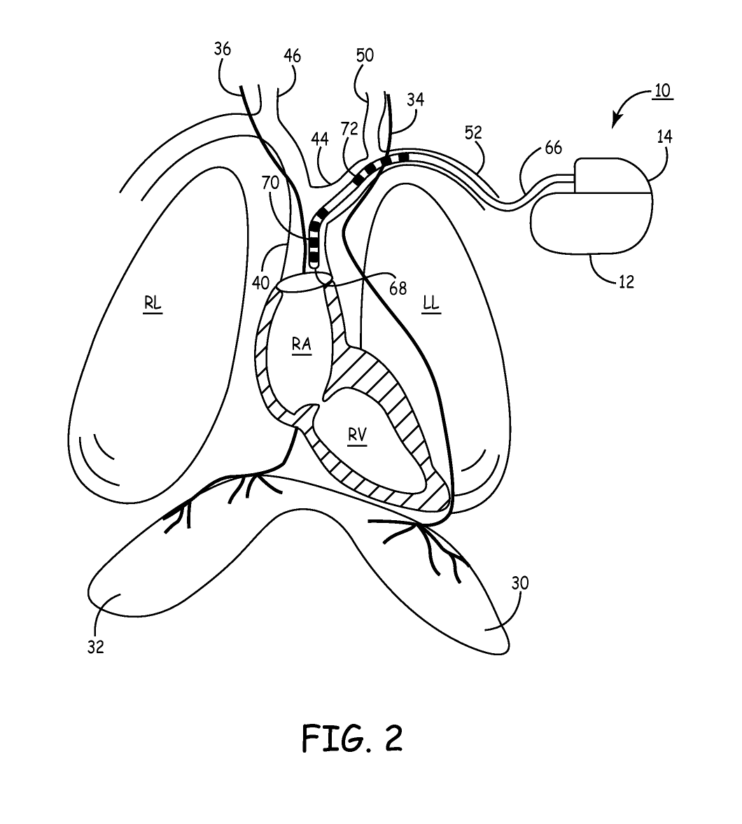

FIG. 2 is a schematic view of an IMD system for delivering phrenic nerve stimulation according to an alternative embodiment. In FIG. 2, the right atrium (RA) and the right ventricle (RV) are shown schematically in a partially cut-away view. The right phrenic nerve 36 extends posteriorly along the SVC 40, the RA and the inferior vena cava (IVC) (not shown in FIG. 2). The left phrenic nerve 34 normally extends along a left lateral wall of the left ventricle (not shown). The SVC 40 enters the RA. A lead 66 is coupled to IMD 10 via connector block 14. Lead 66 carries multiple electrodes, which may be spaced apart into a plurality of distal electrodes 70 located near distal lead tip 68 and a plurality of proximal electrodes 72. The distal tip 68 of lead 66 is advanced into SVC 40 to position distal electrodes 70 for stimulating the right phrenic nerve 36. The proximal electrodes 72 are used to stimulate the left phrenic nerve 34, e.g. along the LIV 44 or junction of the LJV 50 and LSV 52.

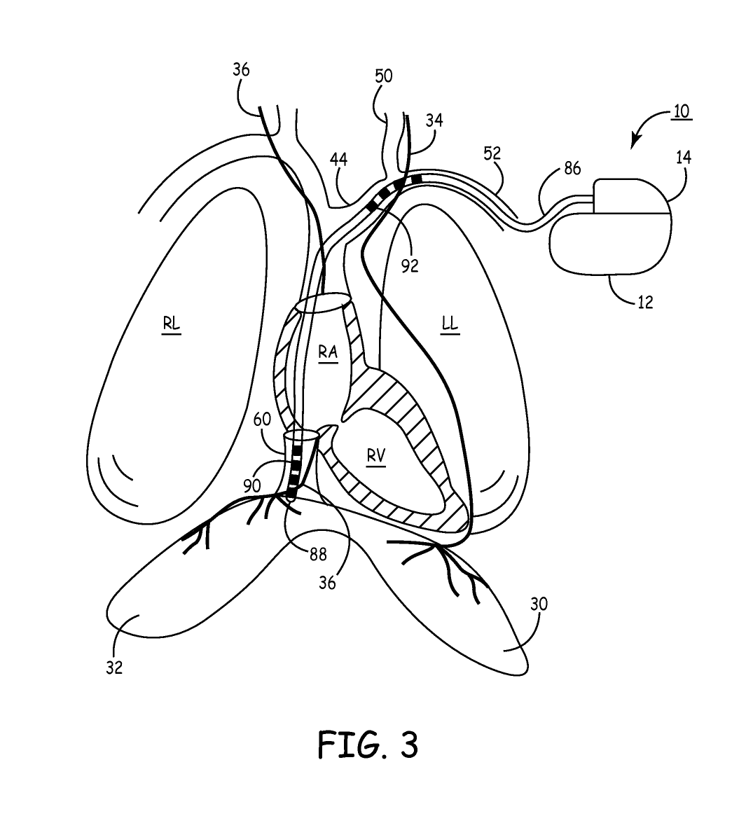

FIG. 3 is a schematic view of an IMD system for delivering phrenic nerve stimulation according to another alternative embodiment. In FIG. 3, the inferior vena cava (IVC) 60, which empties into the RA, is shown schematically. In this embodiment, lead 86 extends from IMD connector block 14 to the IVC 60 to position electrodes 90, carried by lead 86 at or near distal lead tip 88, along the IVC 60 adjacent the right phrenic nerve 36 near the level of the diaphragm, e.g. approximately at the height of the eighth thoracic vertebra (T8) (not shown). Proximal electrodes 92 are positioned proximally along lead 86 for positioning along the LIV 44 or junction of the LJV 50 and LSV 52 for providing stimulation to the left phrenic nerve 34.

Electrodes used for stimulating the right phrenic nerve and electrodes used for stimulating the left phrenic nerve are shown configured along a common lead in FIGS. 1A through 3. In alternative embodiments, it is contemplated that two leads, one for stimulating the left phrenic nerve and one for stimulating the right phrenic nerve, may be provided separately. The housing 12 of IMD 10 may be provided as an indifferent electrode for use in combination with any of the lead-based electrodes shown in FIGS. 1A through 3 for some monitoring purposes. As will be further described below, the electrodes included in an IMD system for delivering a phrenic nerve stimulation therapy may additionally be used for sensing cardiac electrical signals (EGM) signals and for measuring thoracic impedance signals. In some embodiments, the housing 12 may provide an indifferent electrode for sensing EGM signals, delivering a drive current during thoracic impedance measurements or used in a measurement pair for monitoring thoracic impedance.

It is further recognized that additional leads and electrodes may be included in an IMD system capable of delivering transvenous phrenic nerve stimulation (tvPNS). For example, IMD 10 may be coupled to cardiac leads, which may be subcutaneous leads, transvenous leads positioned in or along a heart chamber, or epicardial leads. IMD 10 may incorporate sensing electrodes along housing 12. IMD 10 may be provided specifically for delivering phrenic nerve stimulation (with associated monitoring of sensed signals for controlling the phrenic nerve stimulation) or may include other therapy delivery capabilities such as cardiac pacing (e.g. for bradycardia pacing, cardiac resynchronization therapy, or anti-tachycardia pacing) cardioversion/defibrillation shocks, drug delivery or the like. As such, the IMD system may include other leads, electrodes and/or catheters not shown in FIGS. A1 through 3 as needed for other IMD functions. In some embodiments, electrodes used for delivering phrenic nerve stimulation could be carried by leads that additionally carry cardiac pacing, sensing and/or defibrillation electrodes. In other embodiments, sensing electrodes carried by cardiac leads may be used for sensing EGM signals to detect inadvertent cardiac capture or cardiac nerve stimulation for use in controlling a phrenic nerve stimulation therapy and during positioning of the phrenic nerve stimulation electrodes.

In FIGS. 1A through 3, IMD 10 is shown in a left pectoral position such that it is the distal electrodes, e.g., electrodes 21a, 70, or 90 that are positioned in operative relation to the right phrenic nerve 36 and the proximal electrodes, e.g., electrodes 21b, 72, or 92, that are positioned in operative relation to the left phrenic nerve 34. Depending on the implanted configuration, a phrenic nerve stimulation lead, e.g. lead 16 or 66, may be positioned entering a vein from a right venous approach such that it is the distal electrodes 21 or 70, that are positioned for left phrenic nerve stimulation and the proximal electrodes 22 or 72 that are positioned for right phrenic nerve stimulation. For example, IMD 10 may be implanted in a pocket along a right pectoral position, along a right or left abdominal position, centrally, or other implant location. The IMD implant location may determine whether it is the proximal electrodes or the distal electrodes that are positioned for stimulating the right or the left phrenic nerves, when the electrodes are all carried by a single phrenic nerve stimulation lead.

For example, a right-sided implantation of IMD 10 could include distal electrodes positioned along the LIV 44 for left phrenic nerve stimulation and proximal electrodes positioned for right phrenic nerve stimulation along the RIV 42 or junction of the RSV 48 and RJV 46. As such, in the methods described hereafter, testing and monitoring for EGM sensing, cardiac capture, and/or non-phrenic nerve capture may involve testing of proximal and/or distal electrodes depending on the particular implant configuration being used.

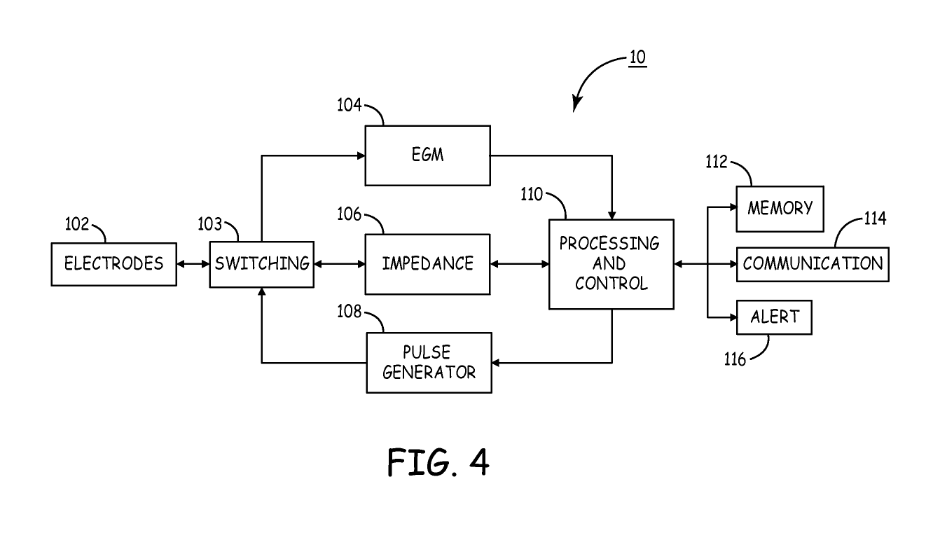

FIG. 4 is a functional block diagram of an IMD that may include any of the leads and implant locations shown in FIGS. 1A through 3. Electrodes 102 are coupled to EGM sensing 104, impedance sensing 106, and pulse generator 108 via switching circuitry 103. Electrodes 102 may correspond to any of the electrodes shown in FIGS. 1 through 3 or other electrodes carried along one or more leads for delivering phrenic nerve stimulation. Electrodes 102 may further include other electrodes available along the IMD housing and any other subcutaneous or cardiac leads coupled to IMD 10.

Electrodes 102 are selected via switching circuitry 103 for coupling to EGM sensing circuitry 104 to sense for the presence of EGM signals on phrenic nerve stimulation electrodes and/or for evidence of inadvertent capture of the heart or cardiac nerves. Electrodes 102 may also be selected in impedance signal drive current and measurement pairs via switching circuitry 103 for monitoring thoracic impedance and the higher frequency cardiac component of the impedance signal by impedance monitoring circuitry 106. An example of such a circuit may be seen with respect to U.S. Pat. No. 5,876,353 issued Mar. 2, 1999 to Riff, and assigned to the assignee of the present invention, the disclosure of which is incorporated by reference in its entirety herein. An exemplary DC impedance signal typically includes a slow, relative large amplitude waveform modulating the signal from respiration as well as a higher frequency signal that happens every heart beat (e.g. two sinusoids with different cycle lengths superimposed). Electrodes 102 are further selected via switching circuitry 103 for delivering phrenic nerve stimulation pulses generated by pulse generator 108.

EGM sensing circuitry 104 is provided for sensing for the presence of an EGM signal on phrenic nerve stimulation electrodes during implantation and during nerve stimulation therapy delivery for detecting a potential risk for cardiac capture. If the electrodes selected for phrenic nerve stimulation are located in close proximity of the heart, nerve stimulation pulses may inadvertently be delivered to the heart, potentially capturing myocardial tissue and inducing arrhythmias. If an EGM signal can be sensed using the electrodes selected for phrenic nerve stimulation, the electrodes may be too close or within the heart. As such, determining that an EGM signal can be sensed using phrenic nerve stimulation electrodes indicates a risk of unintentional cardiac stimulation.

Additionally or alternatively, EGM sensing circuitry 104 is provided for sensing cardiac signals for detecting capture of the heart or a cardiac nerve (e.g. vagus nerve or other sympathetic nerves which may affect heart rate) during phrenic nerve stimulation. In this case, the EGM sensing circuitry may be coupled to any of the phrenic nerve lead electrodes, cardiac electrodes, or subcutaneous electrodes positioned for sensing cardiac EGM or ECG signals such that cardiac events (P-waves or R-waves) may be sensed and used to determine if phrenic nerve stimulation is affecting the rate of these sensed cardiac events.

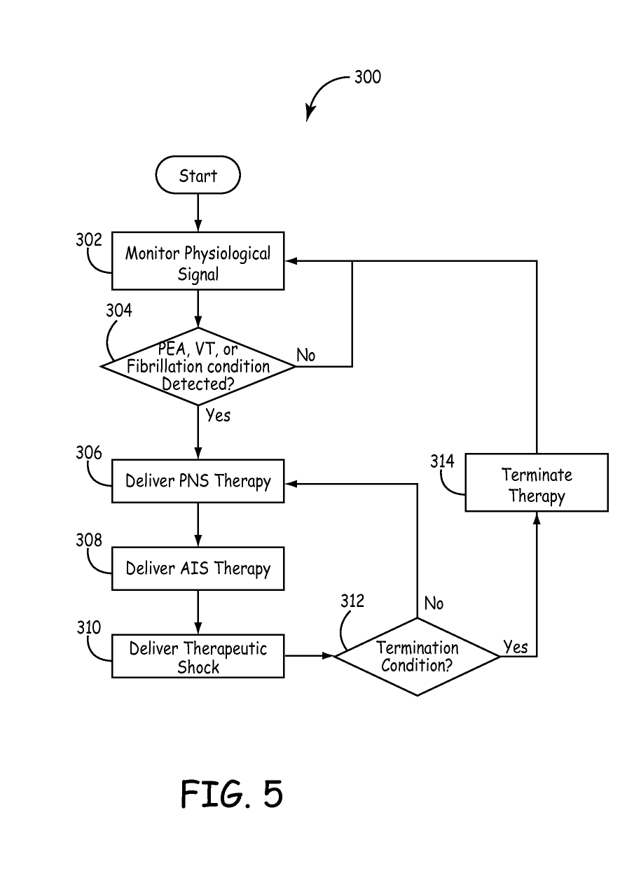

It is recognized that other types of physiological sensors, such as pressure sensors, EMG electrodes or accelerometers may be used for sensing a respiratory response to phrenic nerve stimulation and may be substituted or used in addition to thoracic impedance monitoring or sensing of cardiac contraction. Additionally, oxygen sensors and/or chemical sensors can also be employed such as that which is seen with respect to U.S. Pat. No. 6,198,952 issued Mar. 6, 2001, U.S. Pat. No. 6,666,821 issued Dec. 23, 2003 and assigned to the assignee of the present invention, the disclosure of which is incorporated by reference in its entirety herein. Moreover, computer instructions such as firmware can continuously monitor physiological signals and store data in the memory of the IMD. Sensing data through the lead and storing that data in the memory can be performed independently of the computer instructions or as part of the computer instructions associated with the flow diagrams presented in FIGS. 5-8.

The impedance sensing circuitry 106 includes drive current circuitry and impedance measurement circuitry for monitoring thoracic impedance. The thoracic impedance measurements can be used to select optimal electrodes and stimulation parameters for achieving a desired effect on respiration caused by phrenic nerve stimulation. Respiration involves transport of oxygen from the atmosphere to cells within tissues and carbon dioxide is transported from the tissue to atmosphere. Ventilation which is inspiration and expiration, comprises moving ambient air into and out of the alveoli of the lungs. The impedance sensing circuitry can be used to detect the cardiac component of impedance to determine if there is a heart beat associated with electrical sensing.

Processing and control 110 receives signals from EGM sensing 104 and impedance sensing circuitry 106. In response to received signals processing and control 110 controls delivery of phrenic nerve stimulation by pulse generator 108. Processing and control 110 may be embodied as a programmable microprocessor and associated memory 112. Received signals may additionally include user command signals received by communication circuitry 114 from an external programming device and used to program processing and control 110. Processing and control 110 may be implemented as any combination of an application specific integrated circuit (ASIC), an electronic circuit, a processor (shared, dedicated, or group) and memory that execute one or more software or firmware programs, a combinational logic circuit, or other suitable components that provide the described functionality.

Memory 112 stores data associated with the monitored EGM (or ECG) and impedance signals. Data may be transmitted to an external device by communication circuit 114, which typically includes wireless transmitting and receiving circuitry and an associated antenna for bidirectional communication with an external device. Processing and control 110 may generate reports or alerts that are transmitted by communication circuitry 114.

Alert circuitry 116 may be provided for generating a patient alert signal to notify the patient or a clinician of a condition warranting medical attention. In one embodiment, an alert is generated in response to sensing an EGM signal using the phrenic nerve stimulation electrodes and/or detecting inadvertent capture of the heart or cardiac nerves. EGM sensing or inadvertent capture of the heart or cardiac nerves indicates possible lead dislodgement and risk of cardiac stimulation that may adversely affect heart rate or induce arrhythmias. The patient may be alerted via an audible sound, perceptible vibration, IMD pocket stimulation, or the like and be advised to seek medical attention upon perceiving an alert signal.

Numerous ways exist in which leads can be placed near or directly contact phrenic nerve tissue. An exemplary method for implanting a medical electrical lead near nerve tissue may be seen with respect to US20090276025A1 filed on May 11, 2009, and assigned to the assignee of the present invention, the disclosure of which is incorporated by reference in its entirety herein. Preferably, the lead is placed transvenously. Skilled artisans appreciate that the lead can also be placed transvascularly. Placement of the lead can occur using the technique described herein or other suitable methods for placement of a lead 22 near nerve tissue.

After the lead has been properly placed proximate the phrenic nerve(s), physiological signals are continuously monitored. Physiological data is sensed through electrodes and passed to the A/D converter and then to the microprocessor in the implantable medical device. Typically, computer instructions such as firmware continuously monitor physiological signals and store data in the memory of the IMD. Sensing data through the lead and storing that data in the memory can be performed independently of the computer instructions or as part of the computer instructions.

Flow diagrams presented in FIGS. 5-7 disclose methods 300-500 that reduce or eliminate suspected PEA by delivering timed electrical stimulation. Method 300 is presented in the flow diagram of FIG. 5. Method 300 could be implemented for patients that experience long duration fibrillation episodes.

At block 302, physiological signals from a patient are monitored through IMD 10. In particular, physiological signals such as EGM or cardiac and thoracic impedance or other electrical responses from tissue are sensed by electrodes on one or more leads. The physiological signals are sent to an A/D converter that converts the analog signals to digital signals in IMD 10. The digital signals are then sent to the microprocessor of the IMD 10 for further signal processing and determinations as set forth in the flow diagrams.

At block 304, a determination is made as to whether a cardiac condition is present in a patient. Exemplary conditions that are checked by the microprocessor include PEA, VT and/or a fibrillating heart. U.S. Pat. No. 5,620,468 to Mongeon et al provides an example of a means in which a fibrillating heart rate is detected and determined to exist, U.S. Pat. No. 8,036,742 to Sullivan et al provides an example of a means in which suspected PEA is detected or determined to exist, U.S. Pat. No. 7,474,916 et al provides an example of a means in which VT is detected and determined to exist, all of which are incorporated by reference, in its entirety. Suspected presence of PEA may occur in a number of different situations. For example, assume that in response to detection of VF, a shock is delivered to cardiac tissue. IMD 10, such as an ICD, is configured to pace with a number of intervals to detect of 30/40 beats in the absence of intrinsic electrical activity. PEA can be suspected to be present if, after pacing the cardiac tissue, depolarizations and repolarizations dwindle over time. PEA presence is also suspected to be present if cardiac output decreases. Cardiac output can be determined through the use of a hemodynamic sensor placed in the RV in combination with one or more electrical sensors on a lead used to sense the R wave and/or the L wave. Alternatively, if the number of beats counted over the same time period by the hemodynamic sensor does not substantially match the number of beats counted by the electrical sensor, PEA is likely present. Yet another means for suspecting presence of PEA is lack of a detectable pulse in addition to the EGM.

If no suspected PEA, VT or fibrillation condition is detected, the NO path returns to block 302 in which physiological conditions are continued to be monitored using any means known in the art. If PEA, VT or a fibrillation condition are determined to be present, IMD 10 automatically generates electrical stimulation (e.g. electrical pulses) that is delivered through one or more electrodes on a medical electrical lead to phrenic nerve tissue at block 306. Electrical stimulation parameters associated with delivering electrical stimulation through the lead to phrenic nerve tissue can include at least one or more of current amplitude, voltage, frequency, and/or pulse width. In one or more embodiments, current amplitude can be in the range of about 2 to about 20 mA. Voltage can be in the range of about 1 volt to about 8 volts. Frequency can be in the range of about 20 to 100 Hz. Pulse width can be in the range of about 20 to 400 microseconds (μs). Delivery of PNS is timed to occur at the end of an expiration cycle, which induces artificial inspiration. Delivery of PNS can be extended (e.g. up to 10 seconds) to cause the diaphragm to sufficiently expand to create a deep breath.

Immediately after taking a deep breath, a cough is elicited from the patient in response to electrical stimulation being delivered to direct abdominal muscle stimulation (e.g. abdominal/intercostal tissue stimulation (AIS)) or spinal cord stimulation at block 308. AIS stimulation parameters can include current amplitude in the range of about 2 to about 20 mA. Voltage can be in the range of about 1 volt to about 8 volts. Frequency can be in the range of about 20 to 100 Hz. Pulse width can be in the range of about 50 to 1000 microseconds (us). In one or more embodiments, the AIS electrical stimulation could have an amplitude=5 volts, pulse duration 1000 us, and frequency 100 Hz.

A cough can move oxygenated blood out of the thorax. Moving oxygenated blood out of thorax has been associated with a reduction in the size of the ventricles and perfusion of the coronary arteries. Reduction in cardiac size combined with improved perfusion can improve defibrillation efficacy thereby improving return to spontaneous circulation post-shock.

Sequentially combining phrenic nerve stimulation with abdominal/intercostal stimulation (AIS) moves oxygenated blood out of the thorax, reduces the size of the ventricles, and perfuses the coronaries. AIS therapy involves delivering electrical stimuli in pulses through the one or more electrodes to the surrounding tissue. Pacing parameters can be customized for each patient during implantation of the IMD 10.

At block 310, a therapeutic shock is delivered to cardiac tissue following the PNS and AIS therapies. Exemplary therapeutic shock is a bi-phasic shock pulse of up to 35 joules that can last about 4 ms for the first phase and 4 ms for the second phase. Initiation and/or termination of the therapeutic shock can depend on high voltage shock pathway resistance. Termination of the shock pulse, controlled by a voltage level circuit, is determined by the sensing rate after a shock, the heart rate slows down to normal.

At block 312, a determination is made as to whether a termination condition for one or more of the therapies is present. A termination condition is typically predetermined and saved into the memory of IMD 10. For example, one termination condition can be terminating all therapies once therapeutic shock has successfully resulted in a beating heart that is determined by the physiologic signals, for example a combination of EGM and cardiac impedance.

Alternatively, one or more therapies can be terminated based on one or more conditions. For example, PNS therapy can be terminated after an inspiration cycle (e.g. deep breath.) has been detected. Alternatively, PNS can be terminated after a certain amount of time has expired.

After delivery has begun on at least one therapy, a termination routine or computer instructions can continuously check to determine if a termination condition is met. Each termination routine can operate independently of the computer instructions embodied in the flow diagrams presented herein. The YES path terminates therapy at block 314 and returns to monitoring physiological conditions at block 302.

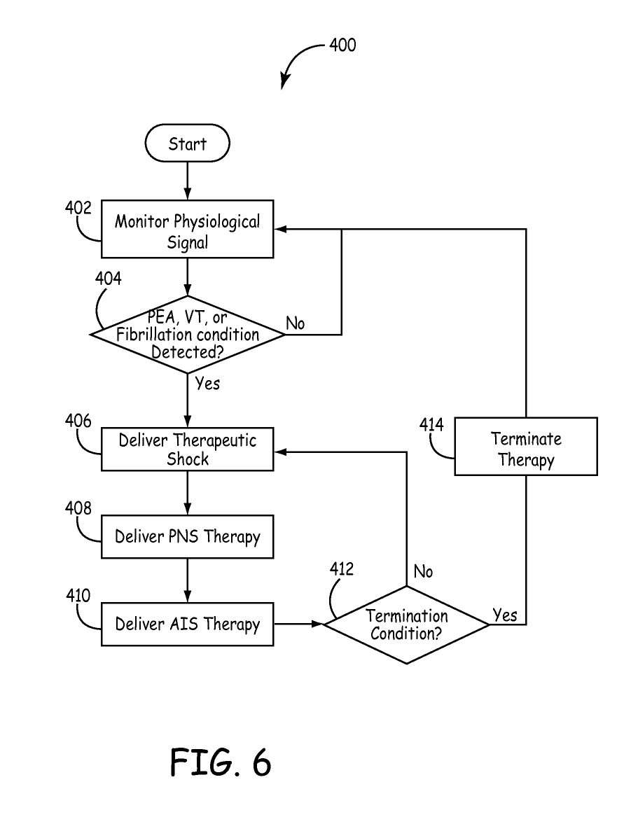

FIG. 6 is a flow diagram that depicts method 400 that assists a patient in recovery of a cardiovascular event where PNS and AIS therapies are used to assist in post-shock hemodynamic recovery. At block 402, physiological signals from a patient are monitored through IMD 10. At block 404, a determination is made as to whether a cardiac condition (e.g. PEA, VT or a fibrillation) is present in a patient. If no cardiac condition is present, the NO path returns to continuous monitoring at block 402. If a fibrillation condition is present, a therapeutic shock is delivered to cardiac tissue at block 406. At block 408, PNS therapy is delivered at the end of an expiration cycle to cause expansion of the diaphragm to allow inspiration (e.g. takes a deep breath).

IMD 10 automatically generates electrical stimulation (e.g. electrical pulses) that is delivered through one or more electrodes on a medical electrical lead to phrenic nerve tissue at block 406. Timed delivery of electrical stimulation to phrenic nerve tissue can cause movement of the diaphragm that results in inspiration or expiration. Immediately after taking a deep breath, a cough is elicited from the patient in response to electrical stimulation being delivered to direct abdominal muscle stimulation (e.g. abdominal/intercostal tissue) or spinal cord stimulation at block 410, which prompts the patient to cough. At block 412, a determination is made as to whether a termination condition for one or more of the therapies is present. For example, if VF has terminated, but PEA is detected, shock therapies can be terminated, but PNS and AIS therapies could continue until a return of an adequate heart beat is detected. The YES path terminates therapy at block 414 and returns to monitoring physiological conditions at block 402.

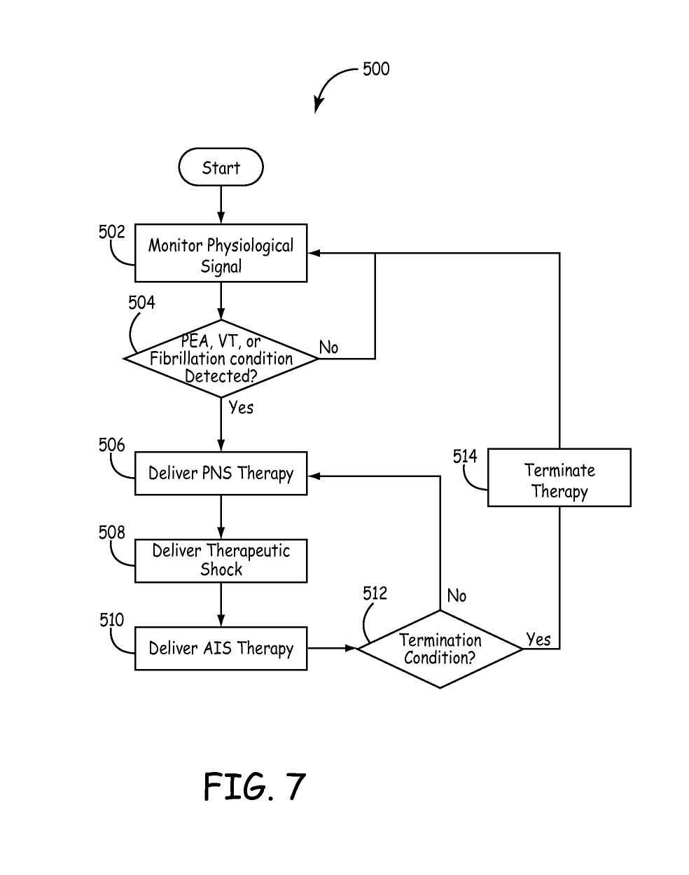

FIG. 7 is yet another flow diagram of method 500. At block 502, physiological signals from a patient are monitored through IMD 10. At block 504, a determination is made as to whether a cardiac condition is present in a patient. If a cardiac condition is not present, the NO path returns to monitoring physiological signals. If PEA, VT or a fibrillation condition are determined to be present, IMD 10 automatically generates electrical stimulation (e.g. electrical pulses) that is delivered through one or more electrodes on a medical electrical lead to phrenic nerve tissue at block 506. Timed delivery of electrical stimulation to phrenic nerve tissue can cause movement of the diaphragm that results in inspiration or expiration. At block 508, immediately after taking a deep breath, a therapeutic shock is delivered to cardiac tissue.

At block 510, a cough is elicited from the patient in response to electrical stimulation being delivered directly to abdominal muscle (e.g. abdominal/intercostal tissue) or spinal cord stimulation. At block 512, a determination is made as to whether a termination condition for one or more of the therapies is present. The YES path terminates therapy at block 514 and returns to monitoring physiological conditions at block 502. The NO path returns to continued therapy delivery at block 506.

Thus, a system and method for a timed therapy delivery system for treatment of PEA have been presented in the foregoing description with reference to specific embodiments. It is appreciated that various modifications to the referenced embodiments may be made without departing from the scope of the disclosure as set forth in the following claims. For example, other embodiments contemplate the AIS therapy being optional or not required for the therapy.

Additionally, skilled artisans appreciate that eliciting a cough from a patient may entail obtaining abdominal stimulation threshold pacing data while implanting the IMD 10 in a patient. To elicit a cough, one or more electrodes associated with a medical electrical lead is placed near or to thoracic spinal nerve tissue. Intensity and duration of the electrical stimulation to the thoracic spinal nerve in order to induce a forced contraction of the intercostal muscle innervated by the thoracic spinal nerve to produce a cough. In one or more embodiments, current amplitude can be in the range of about 2 to about 20 mA. The disclosure of U.S. Provisional Application No. 61/640,464 is incorporated herein by reference in its entirety.

1. A system comprising:
a phrenic nerve electrode, a defibrillation electrode, spatially separated from one another and all coupled to an implantable pulse generator, the phrenic nerve electrode for pacing phrenic nerve tissue and a defibrillation electrode to deliver high voltage therapy to the heart;
means for delivering high voltage therapy through an electrode on to a fibrillating heart; and
in response to delivering high voltage therapy, delivering electrical stimulation through an electrode proximate phrenic nerve tissue.

2. The system of claim 1, wherein the electrical stimulation delivered through the proximate phrenic nerve electrode proximate phrenic nerve tissue occurs during expiration.

3. The system of claim 1, wherein the electrical stimulation delivered through the electrode proximate phrenic nerve tissue occurs at an end of the expiration.

4. The system of claim 1, further comprising:
means for causing artificially induced expiration in response to delivering electrical stimulation through an electrode proximate phrenic nerve tissue.

5. The system of claim 1, further comprising:
means for causing artificially induced inspiration in response to delivering electrical stimulation through an electrode proximate phrenic nerve tissue.

6. The system of claim 1, further comprising:
means for reducing ventricular dilation in response to delivering electric stimulation to phrenic nerve tissue.

7. The system of claim 1, further comprising:
means for increasing perfusion in response to delivering electric stimulation to phrenic nerve tissue.

8. The system of claim 1, further comprising:
means for delivering electrical stimulation through an electrode proximate one of abdominal and intercostal tissue in response to delivering electrical stimulation to phrenic nerve tissue.

9. The system of claim 8 further comprising:
means for eliciting a cough in response to electrical stimulation of one of abdominal and intercostal tissue.

10. The system of claim 9 further comprising:
means for moving oxygenated blood out of a thorax in response to the cough.

11. The system of claim 1, wherein the electrical stimulation delivered through the electrode proximate phrenic nerve tissue occurs at an end of inspiration.

12. The system of claim 1, wherein the electrical stimulation delivered through the electrode proximate phrenic nerve tissue occurs during inspiration.

13. The system of claim 1, wherein the expiration is followed by an induced artificial inspiration.

14. A method of reducing of pulseless electrical activity (PEA) through an electrode for pacing and sensing phrenic nerve tissue and a defibrillation electrode to deliver high voltage therapy to the heart, the method comprising:
placing an electrode proximate phrenic nerve tissue;
delivering high voltage therapy through an electrode to a fibrillating heart; and
in response to delivering high voltage therapy, delivering electrical stimulation through an electrode proximate phrenic nerve tissue.

15. The method of claim 14, wherein the electrical stimulation delivered through the electrode proximate phrenic nerve tissue occurs during expiration.

16. The method of claim 14, wherein the electrical stimulation delivered through the electrode proximate phrenic nerve tissue occurs at an end of the expiration.

17. The method of claim 14, further comprising causing artificially induced expiration in response to delivering electrical stimulation through an electrode proximate phrenic nerve tissue.

18. The method of claim 14, further comprising causing artificially induced inspiration in response to delivering electrical stimulation through an electrode proximate phrenic nerve tissue.

19. The method of claim 14, further comprising reducing ventricular dilation in response to delivering electric stimulation to phrenic nerve tissue.

20. The method of claim 14, further comprising increasing perfusion in response to delivering electric stimulation to phrenic nerve tissue.

21. The method of claim 14, further comprising delivering electrical stimulation through an electrode proximate one of abdominal and intercostal tissue in response to delivering electrical stimulation to phrenic nerve tissue.

22. The method of claim 21 further comprising eliciting a cough in response to electrical stimulation of one of abdominal and intercostal tissue.

23. The method of claim 22 further comprising moving oxygenated blood out of a thorax in response to eliciting the cough.

24. The method of claim 14, wherein the electrical stimulation delivered through the electrode proximate phrenic nerve tissue occurs at an end of inspiration.

25. The method of claim 14, wherein the electrical stimulation delivered through the electrode proximate phrenic nerve tissue occurs during inspiration.

26. The method of claim 14, wherein the expiration is followed by an induced artificial inspiration.

27. A system comprising:
a phrenic nerve electrode, a defibrillation electrode, and one or more electrodes connected to one of abdominal or intercostal tissue spatially separated from one another and all coupled to an implantable pulse generator, the phrenic nerve electrode for pacing phrenic nerve tissue and a defibrillation electrode to deliver high voltage therapy to the heart;
means for delivering high voltage therapy through an electrode on to a fibrillating heart;
in response to delivering high voltage therapy, delivering electrical stimulation through an electrode proximate phrenic nerve tissue; and
in response to delivering electrical stimulation through an electrode proximate phrenic nerve tissue, delivering one of abdominal/intercostal tissue stimulation (AIS) or spinal cord stimulation (SCS) through the one or more electrodes.

28. The system of claim 27, wherein a cough is elicited from a patient in response to delivery of one of AIS and SCS.

29. A system comprising:
a phrenic nerve electrode, a defibrillation electrode, and one or more electrodes connected to at least one of abdominal or intercostal tissue spatially separated from one another and all coupled to an implantable pulse generator, the phrenic nerve electrode for pacing phrenic nerve tissue and a defibrillation electrode to deliver high voltage therapy to the heart;
means for delivering phrenic nerve stimulation through an electrode proximate phrenic nerve tissue;
delivering high voltage therapy through the defibrillation electrode; and
in response to delivering high voltage therapy, delivering electrical stimulation through an electrode proximate one of abdominal/intercostal tissue stimulation (AIS) or spinal cord stimulation (SCS).

30. The system of claim 29, wherein a cough is elicited from a patient in response to delivery of one of AIS and SCS.
